(19) United States
(12) Patent Application Publication
(10) Pub. No.: US 2006/0241371 A1
(43) Pub. Date: Oct. 26, 2006

(54) Method and system to correct motion blur in time-of-flight sensor systems

(75) Inventors: Abbas Rafii, Palto Alto, CA (US); Salih Burak Gokturk, Mountain View, CA (US)

Correspondence Address: Canesta, Inc., 440 N. Wolfe Road, Sunnyvale, CA, 94085, US

(73) Assignee: Canesta, Inc.

(21) Appl. No.: 11/349,312

(22) Filed: Feb. 6, 2006

Related U.S. Application Data
(60) Provisional application No. 60/650,919, filed on Feb. 8, 2005.

(51) Int. Cl. A61B 5/05

(52) U.S. Cl. 600/407

(57) ABSTRACT

A method and system corrects motion blur in time-of-flight (TOF) image data in which acquired consecutive images may evidence relative motion between the TOF system and the imaged object or scene. Motion is deemed global if associated with movement of the TOF sensor system, and motion is deemed local if associated with movement in the target or scene being imaged. Acquired images are subjected to global and then to local normalization, after which coarse motion detection is applied. Correction is made to any detected global motion, and then to any detected local motion. Corrective compensation results in distance measurements that are substantially free of error due to motion-blur.

### RELATION TO PENDING APPLICATIONS

Priority is claimed to co-pending U.S. provisional patent application Ser. No. 60/650,919 filed 8 Feb. 2005, entitled “A Method for Removing the Motion Blur of Time of Flight Sensors”.

### FIELD OF THE INVENTION

The invention relates generally to camera or range sensor systems including time-of-flight (TOF) sensor systems, and more particularly to correcting errors in measured TOF distance (motion blur) resulting from relative motion between the system sensor and the target object or scene being imaged by the system.

### BACKGROUND OF THE INVENTION

Electronic camera and range sensor systems that provide a measure of distance from the system to a target object are known in the art. Many such systems approximate the range to the target object based upon luminosity or brightness information obtained from the target object. However such systems may erroneously yield the same measurement information for a distant target object that happens to have a shiny surface and is thus highly reflective, as for a target object that is closer to the system but has a dull surface that is less reflective.

A more accurate distance measuring system is a so-called time-of-flight (TOF) system. FIG. 1 depicts an exemplary TOF system, as described in U.S. Pat. No. 6,323,942 entitled CMOS-Compatible Three-Dimensional Image Sensor IC (2001), which patent is incorporated herein by reference as further background material. TOF system 100 can be implemented on a single IC 110, without moving parts and with relatively few off-chip components. System 100 includes a two-dimensional array 130 of pixel detectors 140, each of which has dedicated circuitry 150 for processing detection charge output by the associated detector. In a typical application, array 130 might include 100×100 pixels 230, and thus include 100×100 processing circuits 150. IC 110 also includes a microprocessor or microcontroller unit 160, memory 170 (which preferably includes random access memory or RAM and read-only memory or ROM), a high speed distributable clock 180, and various computing and input/output (I/O) circuitry 190. Among other functions, controller unit 160 may perform distance to object and object velocity calculations.

Under control of microprocessor 160, a source of optical energy 120 is periodically energized and emits optical energy via lens 125 toward an object target 20. Typically the optical energy is light, for example emitted by a laser diode or LED device 120. Some of the emitted optical energy will be reflected off the surface of target object 20, and will pass through an aperture field stop and lens, collectively 135, and will fall upon two-dimensional array 130 of pixel detectors 140 where an image is formed. In some implementations, each imaging pixel detector 140 captures time-of-flight (TOF) required for optical energy transmitted by emitter 120 to reach target object 20 and be reflected back for detection by two-dimensional sensor array 130. Using this TOF information, distances Z can be determined.

Emitted optical energy traversing to more distant surface regions of target object 20 before being reflected back toward system 100 will define a longer time-of-flight than radiation falling upon and being reflected from a nearer surface portion of the target object (or a closer target object). For example the time-of-flight for optical energy to traverse the roundtrip path noted at t1 is given by t1=2·Z1/C, where C is velocity of light. A TOF sensor system can acquire three-dimensional images of a target object in real time. Such systems advantageously can simultaneously acquire both luminosity data (e.g., signal amplitude) and true TOF distance measurements of a target object or scene.

As described in U.S. Pat. No. 6,323,942, in one embodiment of system 100 each pixel detector 140 has an associated high speed counter that accumulates clock pulses in a number directly proportional to TOF for a system-emitted pulse to reflect from an object point and be detected by a pixel detector focused upon that point. The TOF data provides a direct digital measure of distance from the particular pixel to a point on the object reflecting the emitted pulse of optical energy. In a second embodiment, in lieu of high speed clock circuits, each pixel detector 140 is provided with a charge accumulator and an electronic shutter. The shutters are opened when a pulse of optical energy is emitted, and closed thereafter such that each pixel detector accumulates charge as a function of return photon energy falling upon the associated pixel detector. The amount of accumulated charge provides a direct measure of round-trip TOF. In either embodiment, TOF data permits reconstruction of the three-dimensional topography of the light-reflecting surface of the object being imaged.

Some systems determine TOF by examining relative phase shift between the transmitted light signals and signals reflected from the target object. Detection of the reflected light signals over multiple locations in a pixel array results in measurement signals that are referred to as depth images. U.S. Pat. No. 6,515,740 (2003) and U.S. Pat. No. 6,580,496 (2003) disclose respectively Methods and Systems for CMOS-Compatible Three-Dimensional Imaging Sensing Using Quantum Efficiency Modulation. FIG. 2A depicts an exemplary phase-shift detection system 100′ according to U.S. Pat. No. 6,515,740 and U.S. Pat. No. 6,580,296. Unless otherwise stated, reference numerals in FIG. 2A may be understood to refer to elements identical to what has been described with respect to the TOF system of FIG. 1

In FIG. 2A, an exciter 115 drives emitter 120 with a preferably low power periodic waveform, producing optical energy emissions of perhaps a few hundred MHz with 50 mW or so peak power. The optical energy detected by the two-dimensional sensor array 130 will include amplitude or intensity information, denoted as “A”, as well as phase shift information, denoted as Φ. As depicted in exemplary waveforms in FIGS. 2B, 2C, 2D, the phase shift information varies with distance Z and can be processed to yield Z data. For each pulse or burst of optical energy transmitted by emitter 120, a three-dimensional image of the visible portion of target object 20 is acquired, from which intensity and Z data is obtained (DATA′). Further details as to implementation of various embodiments of phase shift systems may be found in the two referenced patents.

Many factors, including ambient light, can affect reliability of data acquired by TOF systems. As a result, in some TOF systems the transmitted optical energy may be emitted multiple times using different systems settings to increase reliability of the acquired TOF measurements. For example, the initial phase of the emitted optical energy might be varied to cope with various ambient and reflectivity conditions. The amplitude of the emitted energy might be varied to increase system dynamic range. The exposure duration of the emitted optical energy may be varied to increase dynamic range of the system. Further, frequency of the emitted optical energy may be varied to improve the unambiguous range of the system measurements.

In practice, TOF systems may combine multiple measurements to arrive at a final depth image. But if there is relative motion between system 100 and target object 20 while the measurements are being made, the TOF data and final depth image can be degraded by so-called motion blur. For example, while acquiring TOF measurements, system 100 may move, and/or target object 20 may move, or may comprise a scene that include motion. Motion blur results in distance data that is erroneous, and thus yields a final depth image that is not correct.

What is needed is a method and system to detect and compensate for motion blur in TOF systems.

The present invention provides such a method and system.

### SUMMARY OF THE PRESENT INVENTION

The present invention provides a method and system to detect and remove motion blur from final depth images acquired using TOF systems. The invention is preferably implemented in software executable by the system microprocessor, and carries out of the following procedure. Consecutive depth images I1, I2, I3 . . . In are acquired by the system and are globally normalized and then locally normalized. The thus-processed images are then subjected to coarse motion detection to determine the presence of global motion and/or local motion. If present, global motion and local motion are corrected and a final image in which motion blur has been substantially compensated for if not substantially eliminated results.

Other features and advantages of the invention will appear from the following description in which the preferred embodiments have been set forth in detail, in conjunction with their accompanying drawings.

### BRIEF DESCRIPTION OF THE DRAWINGS

FIG. 1 is a block diagram depicting a time-of-flight three-dimensional imaging system as exemplified by U.S. Pat. No. 6,323,942, according to the prior art;

FIG. 2A is a block diagram depicting a phase-shift three-dimensional imaging system as exemplified by U.S. Pat. No. 6,515,740 and U.S. Pat. No. 6,580,496, according to the prior art;

FIGS. 2B, 2C, 2D depict exemplary waveform relationships for the block diagram of FIG. 2A, according to the prior art;

FIG. 3 is a block diagram depicting a time-of-flight three-dimensional imaging system including de-blur compensation, according to the present invention, and

FIG. 4 is block diagram showing a preferred method of de-blurring data from a TOF system, according to the present invention.

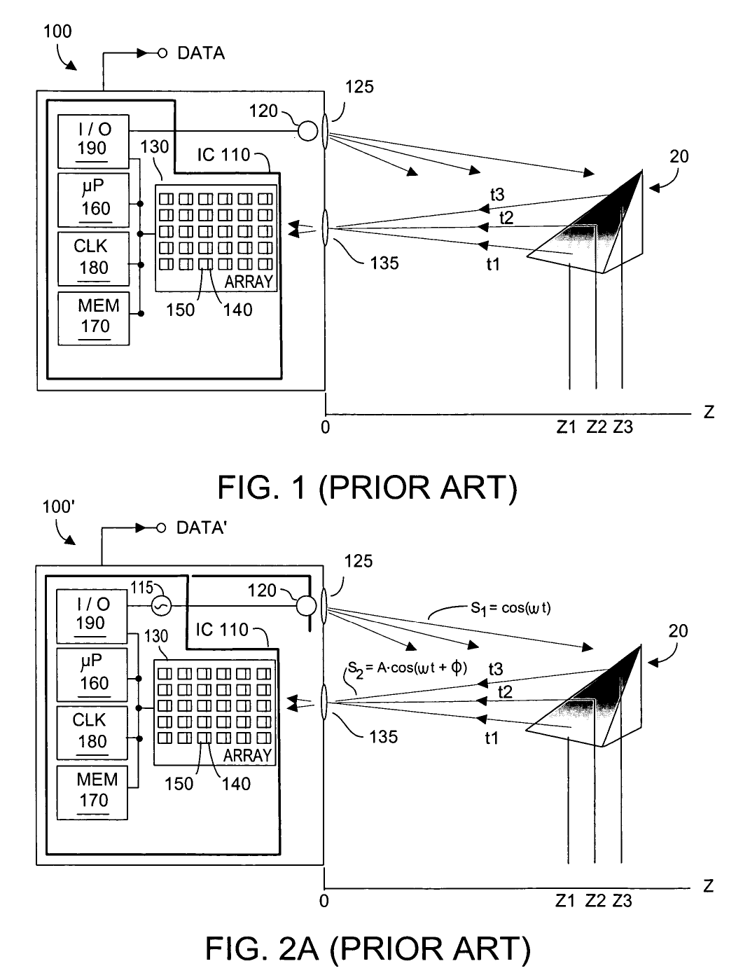

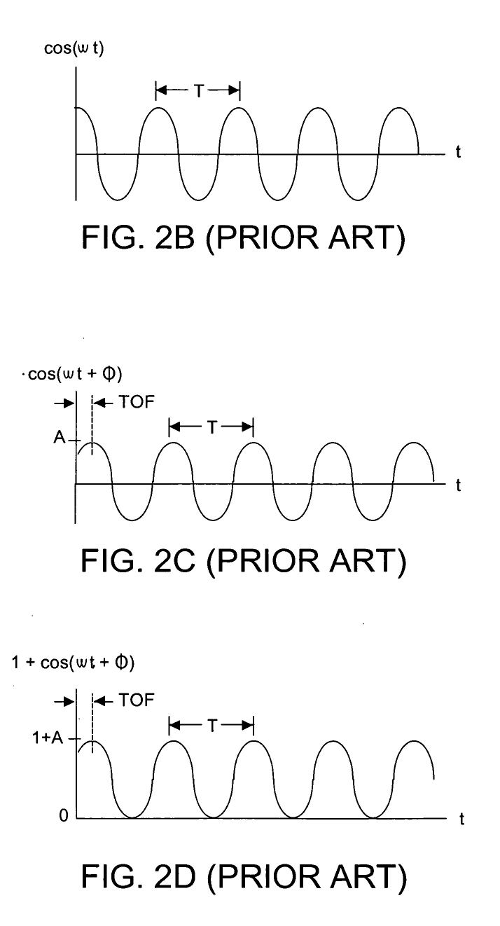

### DETAILED DESCRIPTION OF THE PREFERRED EMBODIMENT

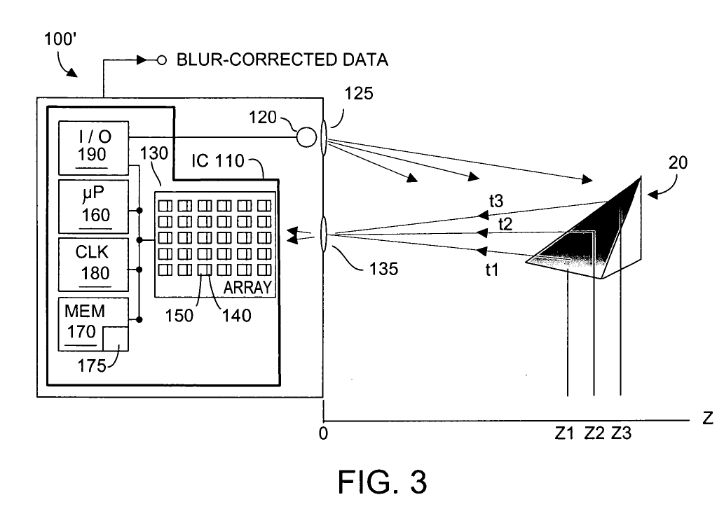

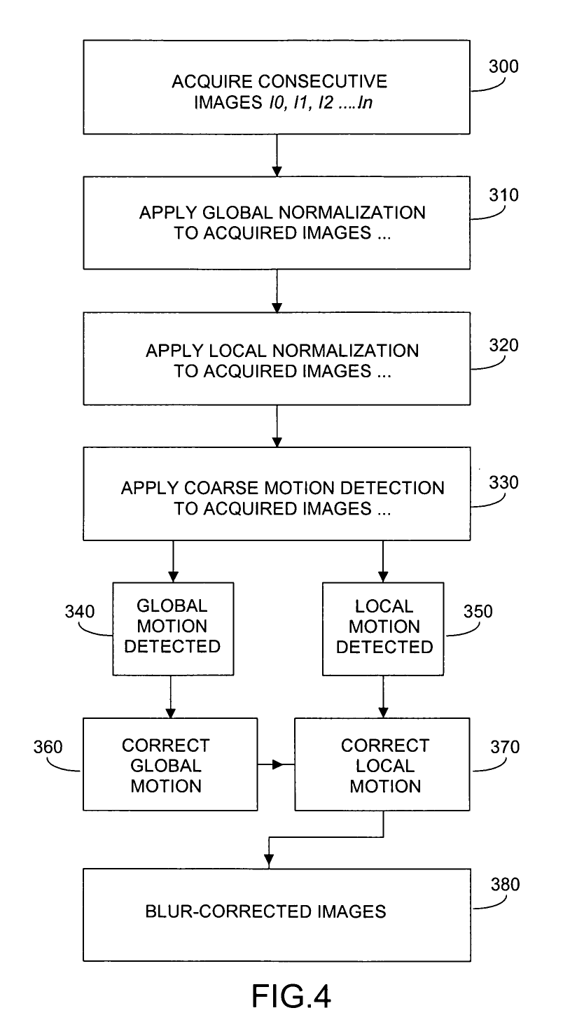

FIG. 3 depicts a system 100′ that includes a software routine or algorithm 175 preferably stored in a portion of system memory 170 to implement the present invention. Routine 175 may, but need not be, executed by system microprocessor 160 to carryout the method steps depicted in FIG. 4, namely to detect and compensate for relative motion error in depth images acquired by system 100′, to yield corrected distance data that is de-blurred with respect to such error.

As noted, it usually is advantageous to obtain multiple data measurements using a TOF system 100′. Thus, microprocessor 160 may program via input/output system 190 optical energy emitter 120 to emit energy at different initial phases, for example to make system 100′ more robust and more invariant to reflectivity of objects in scene 20, or to ambient light level effects in the scene. If desired, the length (exposure) and/or frequency of the emitter optical energy can also be programmed and varied. Each one of the acquired data measurements produces a depth image of the scene. However the acquired scene images may have substantially different brightness levels since the exposure and/or the initial phase of the emitted optical energy can directly affect the acquired intensity levels.

In practice, each of the detected images may take tens of milliseconds to acquire. This is a sufficiently long time period during which motion could occur in the scene 20 being imaged and/or movement of system 100′ relative to the scene. When there is motion in the scene, it is likely that each of these images contains measurements from objects with different depths. As a result, a depth data value obtained by system 100′ from the combination of these images could easily be erroneous, and would the resultant final depth image. It is the function of the present invention, executable routine 175, to normalize the detected data in a sequence of acquired depth images, and then detect and correct for relative motion between the acquisition system and the target object or scene. The present invention results in final depth images that are substantially free of motion blur.

Referring to FIG. 4, an overview of the present invention will be given, followed by specific embodiments of implementation. Method step 300 represents the normal acquisition by system 100′ of a series of measurements or depth images denoted I0, I1, I2, I3 . . . In. As noted, for a variety of reasons relative motion may be present between successively acquired images, for example between I1 and I2, between I2 and I3, and so forth.

According to the present invention, preferably each image is initially normalized at method steps 310, 320 to compensate for motion between adjacent images, e.g., between images I0 and I1, between images I1 and I2, and so on. Initially one of the acquired images is selected as a reference image. Without limitation, let the first acquired image I0 be the reference image, although another of the images could instead be used.

Before trying to detect the presence of motion between each image I1, I2, . . . In and the reference image I0, the sequence of images I0, I1, I2, I3 . . . In are normalized, preferably using two types of normalization. At step 310, global normalization compensates for the differences in the images due to global settings associated with system 100′ (but not associated with target object or scene 20). Then at step 320, local normalization is applied as well to compensate for differences associated with target 20 (but not system 100′).

Next, at method step 330 coarse motion detection is applied to determine which pixel detectors 140 in array 130 have captured motion. Method steps 330, 340, 350 serve two functions. First, the nature of the pixel detector-captured motion is categorized in terms of being global motion or local motion. Method step 340 determines whether the motion is global motion, e.g., motion that results from movement of system 100′ or at least movement of sensor array portion 130. Method step 350 determines whether the motion is local motion due to movement in scene 20. Second, the ability of steps 330, 340, 350 to categorize the type of motion improves performance of routines to compensate for the respective type of the motion.

Once the global and/or local characteristic of the motion has been determined, the appropriate motion correction or compensation is carried out at method steps 360, 370. At method step 360, global motion is compensated for over the entire image, after which local motion is compensated for at the pixel detector level. After each of these compensations is applied, the images I0,I1,I2, . . . In should have the same view of the acquired scene, and as such these corrected images can now be combined to generate a depth image that is free of motion blur, as shown by method step 380.

Having broadly described the methodology shown in FIG. 4, specific implementations of the various method steps will now be given.

Referring to globalization method step 310, multiple images (I₀, I₁, I₂, . . . Iₙ) will typically have been captured under different conditions. For instance, the images may be captured with different emitted energy phases, and/or with different exposure durations, and may exhibit different intensity levels. At method step 310 all images I₁, I₂, . . . Iₙ are normalized to have comparable intensity levels with the reference image I₀.

In one embodiment, the mean and the standard deviation of the image I₀ are obtained. Let μ₀ and σ₀ be the mean and standard deviation of the reference image I₀. Let μᵢ and σᵢ be the mean and standard deviation of one of the images Iᵢ where i=1 . . . n. Let Iᵢ(x,y) the intensity value of the image li at pixel location (x,y). Then, the image Iᵢ(x,y) can be normalized to obtain the normalized image Iᵢ^{N}(x,y) as follows:

$${I}_{i}^{N} ( x , y ) = \frac{{I}_{i} ( x , y ) - {μ}_{i}}{{σ}_{i}} \cdot {σ}_{0} + {μ}_{0}$$

As a consequence, the normalized image Iᵢ^{N} has the same mean and standard deviation as the reference image I₀.

Alternatively, normalization can be implemented using histogram based techniques where the density function of the image is estimated. In another embodiment, normalization is implemented using an edge image, assuming here that image edges are preserved regardless of the brightness changes in the scene. An edge image, obtained by an edge detector algorithm can be applied on the input images I₀, I₁, I₂, . . . In, to yield edge images E₀, E₁, E₂, . . . Eₙ, These edge images are provided as an input to method step 339, where motion is detected and then at steps 340, 350, 360, 370 characterized and appropriately compensated for.

Referring now to FIG. 4, step 320, in addition to global normalization, a local normalization around each pixel detector acquiring the image may be required. This normalization can be important during subsequent motion compensation, and preferably the motion compensation procedures can function on a locally normalized image at each pixel detector.

In one embodiment, a methodology similar to the global normalization method carried out at step 310 may be used. In this embodiment the mean and standard deviation normalization, or edge normalization can be applied on image patches (e.g., sub-images), as opposed to being applied to the entire image.

Referring now to the coarse level motion detection steps shown in FIG. 4, the algorithm method to be described preferably are implementable in an embedded platform where a low-power central processing unit is available, for example microprocessor 160.

Method step 330 provides coarse level motion detection to increase the efficiency of the algorithm. What is desired here is the creation of a map Mₖ for each image k=1,2, . . . n, where the map denotes the existence of motion at a particular pixel (x,y) on each image. Each pixel of Mₖ(x,y) is either 0 or 1, where the value of 1 denotes the presence of motion.

In one embodiment, motion between consecutive frames of acquired images is defined as a change between consecutive frames. This can be implemented by examining the normalized images Iᵢ^{N}. More specifically, at every pixel (x,y), the difference to the normalized reference image I₀^{N} is determined. If this difference is greater than a threshold (T), then the map image is assigned a value of 1:

$${M}_{i} ( x , y ) = \{ \begin{matrix}1 & if \vert {I}_{i}^{N} ( x , y ) - {I}_{0}^{N} ( x , y ) \vert \geq T \\ 0 & if \vert {I}_{i}^{N} ( x , y ) - {I}_{0}^{N} ( x , y ) \vert < T\end{matrix}$$

This map can advantageously increase the efficiency of the following steps, where the calculations are only applied to pixels where Mᵢ(x,y)=1.

In method steps 340, 360, global motion compensation compensates for system 100′ motion, more specifically motion in pixel detector array 130, which motion is primarily in the (x-y) plane. It is implicitly assumed that any rotational motion is programmable as finite (x-y) motions.

In one embodiment, a global block matching method is used, in which a large portion of the image is used as the block. The algorithm inputs are the normalized images Iᵢ^{N}, or the edge images Eᵢ from global normalization step.310. The algorithm finds the motion vector (Δx, Δy) where the following function energy function (ε) is minimized:

$${ɛ}_{i} ( Δ x , Δ y ) = {\sum }_{x ∈ I} {\sum }_{y ∈ I} {⌊ {I}_{i}^{N} ( x + Δ x , y + Δ y ) - {I}_{0}^{N} ( x , y ) ⌋}^{2}$$

As such, global block matching essentially carries out an optimization procedure in which the energy function (ε) is calculated at a finite set of (Δx, Δy) values. Then the (Δx, Δy) pair that minimizes the energy function is chosen as the global motion vector.

In another embodiment, the block matching algorithm is improved by a log-search in which the best (Δx, Δy) pair is obtained and then improved by a more local search around the first (Δx, Δy) pair. The iteration continues while, at each iteration, the search area is reduced around the previous match so that a finer motion vector is detected.

In yet another embodiment, global motion is determined using a phase-detection method. For instance, in a TOF system that uses phase shift method to determine distance, if the measurement from symmetric phases (such as 0° and 180°) are not symmetric, the discrepancy is an indication of a local or global motion.

Referring to FIG. 4, step 350, in one embodiment a Lucas-Kanade motion detection algorithm is applied to detect motion at every pixel detector. The method is somewhat analogous to global motion detection, as described above. Optimization will now be based upon the following equation:

$${ɛ}_{i , p} ( Δ x , Δ y ) = {\sum }_{x ∈ {w}_{i , p}} {\sum }_{y ∈ {w}_{i , p}} {⌊ {I}_{i}^{N} ( x + Δ x , y + Δ y ) - {I}_{0}^{N} ( x , y ) ⌋}^{2}$$

In the above equation, optimization is applied on a window w_{i,p} around every pixel (or group of pixel) p of image Iᵢ. The solution to this problem may be carried out using a Lucas-Kanade tracker, which reduces the analysis to the following equation:

$$[ {I}_{x} {I}_{y} ] [ \begin{matrix}Δ x \\ Δ y\end{matrix} ] = [ - {I}_{t} ]$$

In the above equation, Iₓ and I_{y} are the spatial derivatives of the image I in x and y directions respectively. The relationship represents the temporal derivative of the image I, where Δx and Δy are the motion vectors. The pixels in the window w can be used to solve this optimization problem using an appropriate optimization algorithm. Common iterative optimization algorithms can be used to solve for Δx and Δy. In one embodiment, a pyramidal approach is used, where an initial estimate of the motion vector is found using one or more down-sampled versions of the image, and the fine motion is extracted using the image. This approach reduces failure modes such as the locking of an optimization algorithm at a local maximum.

After method step 350 detects local motion, applicable correction or compensation is made at method step 370. Once the motion vector [Δx, Δy] is determined for every pixel p, and every image Iᵢ, motion compensation is readily established by constructing an image Iᵢ₀ for each image Iᵢ:
Iᵢ₀^{N}(x,y)=I^{N}(x+Δx,y+Δy)

Referring now to method step 380, at this juncture all operations between image Ii and I0 may now be carried out using images Iᵢ₀^{N} and I₀^{N}. The result following method step 380 is the construction of a depth image that is substantially free of motion blur.

Implementation of the above-described steps corrects motion blur in a TOF system, for example system 100′. FIG. 4 described normalizing the input images, then detecting the type(s) of motion present, and correcting global motion and local motion. However in some applications, it may not be necessary to carryout each step shown in FIG. 4. For example system 100′ may be used in a factory to image objects moving on a conveyor belt beneath the sensor system. In this example, most of the motion would be global, and there would be little need to apply local motion estimation in arriving at depth images substantially free of motion blur.

Modifications and variations may be made to the disclosed embodiments without departing from the subject and spirit of the invention as defined by the following claims.

What is claimed is:

1. A method of compensating for error measurement in depth images due to relative motion between a system acquiring the images using an array of pixels and a target object being imaged, the method comprising the following steps:
(a) acquiring a sequence of images;
(b) normalizing the acquired said sequence of images relative to a referenced one of said images;
(c) detecting presence of at least one of coarse motion associated with movement of said system, and local motion associated with movement of said target object, in said acquired said sequence of images; and
(d) compensating for at least one of coarse motion and local motion in said acquired said sequence of said images;
wherein images so compensated at step (d) are substantially free of distance error due to said relative motion.

2. The method of claim 1, wherein said system is a time-of-flight system.

3. The method of claim 1, wherein step (b) includes arbitrarily selected one of said images as said reference image.

4. The method of claim 1, wherein step (b) includes normalizing to have comparable intensity levels in said images relative to said reference image.

5. The method of claim 1, wherein step (b) normalizes said images to have a mean and a standard deviation equal to a mean and a standard deviation of said reference image.

6. The method of claim 1, wherein step (b) includes at least one method selected from a group consisting of normalizing said images using edge detection, and normalizing said images using sub-image patches of said images.

7. The method of claim 1, wherein step (b) includes normalizing relative to each pixel in said pixel array.

8. The method of claim 1, wherein step (b) includes normalizing relative to each pixel in said pixel array using at least one method selected from a group consisting of normalizing image mean and standard deviation, normalizing image edges, and normalizing sub-image patches of said images.

9. The method of claim 1, wherein step (c) includes detecting motion between consecutive frames of said images.

10. The method of claim 9, wherein step (c) further includes detecting differences between normalized said images relative to a reference threshold difference.

11. The method of claim 1, wherein step (c) includes matching substantial block portions of said images relative to at least one of normalized said images and detected edges of normalized said images.

12. The method of claim 11, wherein step (c) minimizes a function given by
$${ɛ}_{i , p} ( Δ x , Δ y ) = {\sum }_{x ∈ {w}_{i , p}} {\sum }_{y ∈ {w}_{i , p}} {⌊ {I}_{i}^{N} ( x + Δ x , y + Δ y ) - {I}_{0}^{N} ( x , y ) ⌋}^{2}$$
where movement of said system is in an (x,y) plane, and where Iᵢ^{N} is a normalized image, (Δx, Δy) is a motion vector, where energy function (E) is minimized, and a (Δx, Δy) minimizing (ε) is selected as a global motion vector.

13. The method of claim 12, further including iterating around a first (Δx, Δy) pair obtained in minimizing energy function (ε).

14. The method of claim 1, wherein step (c) includes detecting local motion by applying Lucas-Kanade motion detection on a per pixel basis, where optimization solves an equation:
$${ɛ}_{i , p} ( Δ x , Δ y ) = {\sum }_{x ∈ {w}_{i , p}} {\sum }_{y ∈ {w}_{i , p}} {⌊ {I}_{i}^{N} ( x + Δ x , y + Δ y ) - {I}_{0}^{N} ( x , y ) ⌋}^{2}$$
where optimization is applied optimization is applied on a window w_{i,p} around one of every pixel p and every group of pixels p of image Iᵢ.

15. The method of claim 14, further including solving said equation using a Lucas-Kanade tracker.

16. The method of claim 1, wherein step (d) includes determining a vector [Δx, Δy] for every pixel p and for every image Iᵢ, and compensating by constructing an image Iᵢ₀ for each image Iᵢ: given by Iᵢ₀^{N}(x,y)=Iᵢ^{N}(x+Δx,y+Δy).

17. A de-blurring system to compensate for error measurement in depth images due to relative motion between a system acquiring the images using an array of pixels and a target object being imaged, the de-blurring system comprising:
a microprocessor unit;
memory storing a routine that upon execution by said microprocessor unit carries out the following steps:
(a) normalizing a sequence of images, acquired by said system, relative to a referenced one of said images;
(b) detecting presence of at least one of coarse motion associated with movement of said system, and local motion associated with movement of said target object, in said acquired said sequence of images; and
(c) compensating for at least one of coarse motion and local motion in said acquired said sequence of said images;
wherein images so compensated at step (c) are substantially free of distance error due to said relative motion.

18. The de-blurring system of claim method of claim 17, wherein said system is a time-of-flight system.

19. The de-blurring system of claim 17, wherein step (a) includes normalizing said images to have at least one characteristic selected from a group consisting of (i) said images have comparable intensity levels in said images relative to said reference image, (ii) said images have a mean and a standard deviation equal to a mean and a standard deviation of said reference image, (iii) said images are normalized using edge detection, and (iv) said images are normalized using sub-image patches of said images.

20. The de-blurring system of claim 17, wherein step (b) includes at least one of (i) detecting motion between consecutive frames of said images, (ii) detecting differences between normalized said images relative to a reference threshold difference, and (iii) matching substantial block portions of said images relative to at least one of normalized said images and detected edges of normalized said images.
